(19) United States
(12) Patent Application Publication
(10) Pub. No.: US 2002/0143384 A1
(43) Pub. Date: Oct. 3, 2002

(54) Stent cover and stent

(76) Inventor: Hitoshi Ozasa, Kanagawa (JP)

Correspondence Address: Platon N. Mandros, BURNS, DOANE, SWECKER & MATHIS, L.L.P., P.O. Box 1404, Alexandria, VA, 22313-1404, US

(21) Appl. No.: 10/108,857

(22) Filed: Mar. 29, 2002

(30) Foreign Application Priority Data
Mar. 30, 2001 (JP) 2001-98810

(51) Int. Cl. A61F 2/06

(52) U.S. Cl. 623/1.12

(57) ABSTRACT

A cylindrical stent cover covering a cylindrical stent body wherein open at both ends and longitudinally elongates between the both open ends having an inner side surface and an outer side surface, said stent cover covers the inner side surface and/or outer side surface of said stent body, said stent cover comprise a central part made of a nonporous film and the both ends made of a porous film. When the stent cover use as cover of stent body placed in body duct like the vessel, it provides that maintains lumen in a state of patency over a long period of time and has an effect of preventing restenosis.

### TECHNICAL FIELD

The present invention relates to a stent and a stent cover wherein is used for the stent. More particularly, the present invention relates to a stent having a stent cover(s) capable to maintaining its lumen in a state of patency over a long period of time when it is used as a stent wherein is placed in the body duct such as vessels including blood vessels and bile ducts. And also, the present invention relates a stent cover wherein is used for the stent.

### BACKGROUND ART

Conventionally, in the therapy of angina pectoris, myocardial infarction or the like, it has been performed to permanently implant a metal-made stent in a coronary artery or other vessels to ensure blood flow therein in order to prevent restenosis, which occurs in a high ratio after treating a stenosed portion of coronary artery by percutaneous transluminal coronary angioplasty (PTCA), etc., or apply it to a form of lesion in which satisfactory expansion is not obtained by PTCA. Such a stent generally has a tubular structure made of metal wires that can be reduced in diameter, in the form of mesh, coil or the like. It is inserted into a vessel in a state where it has a reduced diameter by means of a catheter, and in the stenosed portion it is expanded in diameter and indwelled so that it can mechanically support the lumen of the vessel. However, it has been reported that indwelling of such an intravascular stent also caused restenosis due to growth and hyperplasia of a lumen tissue through interstices between the metal wires that constitute the stent.

To solve this problem, attempts have been made to prevent growth/hyperplasia of a tissue into the lumen of a stent by covering the stent with a porous film on the inner side/outer side thereof and fixing the film to the stent to provide the stent with a cover as disclosed in JP 07-24072 A.

However, at end portions of the cover, a turbulent flow tends to be generated in body fluid that flows in the vessel. When the body fluid is blood, the disturbance of blood flow makes blood susceptible to formation of thrombi and in the case of a stent wherein covered by stent cover and indwelled in a blood vessel, the thrombi formed at end portions of the cover will grow to cause thrombotic occlusion, which raises a problem in using a stent covered by stent covers.

### DISCLOSURE OF THE INVENTION

The present invention has been made in consideration of the problems encountered in the conventional technology as described above and an object of the present invention is to provide a stent having a stent cover(s) capable of maintaining a lumen in a state of patency over a long period of time and preventing restenosis when a stent is placed inside body duct such as vessels.

The above-mentioned object will be solved by the following aspects of the present invention.

(1) There is provided a cylindrical stent cover covering a cylindrical stent body wherein open at both ends and longitudinally elongates between the both open ends having an inner side surface and an outer side surface, said stent cover covers said inner side surface and/or outer side surface of the stent body, a central part of said stent cover comprises a nonporous film and both end parts of said stent cover comprise a porous film.

(2) There is provided a stent cover according to (1) in which said porous film of the end parts comprises a nonwoven fabric.

(3) There is provided a stent cover according to (1) or (2) in which said nonporous film of the central part and said porous film of the end parts of the stent cover comprise a material having biocompatibility.

(4) There is provided a stent cover according to any one of (1) to (3) in which said nonporous film of the central part and said porous film of the end parts of the stent cover comprise a material having biodegradability or bioabsorbability.

(5) There is provided a stent cover according to any one of (1) to (4) further in which a drug for preventing restenosis of a stent-implanted body duct or promoting endothelialization at said end part of the stent cover is added to said stent cover.

(6) There is provided a stent cover according to (5) in which said drug has at least one of effects selected from the group consisting of antithrombotic effect, prevention of cell migration, prevention of cell growth, and promotion of endothelial cell growth.

(7) There is provided a stent cover according to (5) or (6) in which said drug is added in a state where it is contained in a gel.

(8) There is provided a stent cover according to (7) in which said gel is inactive to a living organism.

(9) There is provided a stent cover according to (7) or (8) in which said gel has biodegradability or bioabsorbability.

(10) There is provided a stent comprises a stent body wherein open at both ends and longitudinally elongates between the both open ends having an inner side surface and an outer side surface, and a stent cover(s) according to any one of (1) to (9).

(11) There is provided a stent comprises a stent body wherein open at both ends and longitudinally elongates between the both open ends having an inner side surface and an outer side surface, a stent cover according to any one of (1) to (9) wherein covers the outer side surface of the stent body, a porous film wherein covers both end portions of the inner side surface of the stent body but doesn't cover central portion of the inner side surface of the stent body.

### BRIEF DESCRIPTION OF THE DRAWINGS

FIG. 1 is a side elevational perspective view showing a mode of construction of a stent according to the present invention;

FIG. 2 is a cross sectional view taken along line 20-20 of FIG. 1;

FIG. 3 is a cross sectional view taken along line 30-30 of FIG. 1;

FIG. 4 is a perspective view showing a stent body wherein is different in shape with that of FIG. 1;

FIG. 5 is a side elevational perspective view showing the construction of a stent of Comparative Example 1;

FIG. 6 is a side elevational perspective view showing the construction of a stent of Comparative Example 2;

FIG. 7 is a schematic diagram illustrating the manner of implanting a stent in the abdominal aorta of a rabbit;

FIG. 8 is a cross-sectional view showing a pathological tissue of abdominal aorta of a rabbit having implanted therein the stent of Example 2, taken along a plane obtained by cutting a portion in the vicinity of an end part of the stent vertical to the axis of the stent; and

FIG. 9 is a cross-sectional view showing a pathological tissue of abdominal aorta of a rabbit having implanted therein a stent of Comparative Example 1, taken along a plane obtained by cutting a portion in the vicinity of an end part of the stent vertical to the axis of the stent.

### BEST MODE FOR CARRYING OUT THE INVENTION

Hereinafter, the stent cover of the present invention and the stent using the stent cover will be illustrated in more detail with reference to the attached drawings.

In overall shape, the stent cover of the present invention is a hollow cylindrical film form in order to cover the inner side surface and/or outer side surface of a stent body. Hereinafter, a stent body has a cylindrical structure wherein open at both ends and longitudinally elongates between the both open ends having inner side surface and outer side surface, and the cylindrical structure has plurality of cutout part on the side surface thereof communicating inner and outer side surface. The cylindrical structure is constructed so that with deformation of the cutout part, the expansion and contraction in radial direction of the cylindrical structure is possible. In the long axial direction, a central part of the cylindrical stent cover comprises a nonporous film and its end parts, that is, parts on the side of the open ends of the stent body comprise a porous film. Because of being formed by a nonporous film, the central part of the stent cover has low water permeability, whereby cellular infiltration from the body duct to inner luminal side of the stent body can be prevented and growth and hyperplasia of a lumen tissue into inner luminal side of the stent body can be prevented. On the other hand, because of being formed by porous films, each end parts of the stent cover have high water permeability, whereby formation of smooth endothelialized surface from the vascular wall of a part in the vicinity of the stent cover to the surface of the stent cover facing lumen of the stent can be promoted and it is superior in fixation to the tissue.

Here, the ratio of length of the central part to that of end part of the cylindrical stent cover is 100:7400 to 100:2, preferably 100:200 to 100:5, more preferably 100:100 to 100:10, most preferably 100:50 to 100:20. Setting the ratio within the above-mentioned ranges, growth and hyperplasia of a lumen tissue into inner lumen of the stent body can be prevented and endothelialization at the end parts of the stent cover can be sufficiently promoted when the stent cover of the present invention is used as a cover(s) of a stent which is placed in the body duct. As for a length of end part and that of central part of the stent cover, in the case of an ordinary intravascular stent having a length of the cylindrical body in the long axial direction is 10 to 150 mm, setting the length of each end part within the range that is 1 mm or more, and the length of the central part within the range that is 3 mm or more, purpose of the present invention can be achieved sufficiently. Here, in general, two end parts of the stent cover are usually almost identical in length, however it doesn't limited for the length, each end parts may have different length.

A nonporous film forming the central part of the stent cover according to the present invention is a nonporous film prepared from a material that has stretch properties and is readily stretched with a slight stress and undergoes plastic deformation without preventing expansion of the stent body. In consideration of the utility of the present invention, it is preferred that the nonporous film is formed from a material having biocompatibility.

Examples of such a material include polyolefins, polyesters, fluororesins, silcones, polyurethanes, polyamides, polysulfones, polyethers, polyglycolic acids, polylactic acids, polycaprolactones, polyglactins, polygluconic acids, polyhydroxybutyric acids, chondroitin sulfate gels, hyaluronic acid gels, fibrin, celluloses, polyorthoesters, polyhydroxybutyrate valerate, and the like. These may be single substance, or copolymers or mixtures.

It is more preferable that the nonporous film described above is made from a material having biodegradability or bioabsorbability. Among these, examples of such a material include polylactic acids, polycaprolactones, polyglycolic acids, polyglactins, polyhydroxyburyric acids, chondroitin sulfate gels, hyaluronic acid gels, fibrin, celluloses, polyorthoesters, polyhydroxybutyrate valerates, and the like. These may be single substance, or copolymers or mixtures.

The nonporous film described above can advantageously made by a method usually used for making a film or a sheet from the above-mentioned materials.

It is preferable that a porous film that forms end parts of the stent cover according to the present invention, like the nonporous film that forms the central part, comprises a film that has stretch properties and is readily stretched with a slight stress and undergoes plastic deformation without preventing expansion of the stent body. Such a porous film is made of any one of a porous film obtained by blowing a film of a polymer such as polyolefins or polyurethanes, a nonwoven fabric made by melt-blow molding, a similar polymer material, and a woven fabric made from a cellulose fiber, or the like. These porous films can be advantageously obtained by making foamed polymer film, nonwoven fabric or woven fabric from the materials exemplified with respect to the central part of the stent cover by any known method.

It is preferred that the porous film made of the foamed polymer film has a porosity of 1×10¹ to 1×10⁵ cells/cm², particularly preferably 1×10¹ to 1×10⁴ cells/cm². In addition, it is preferred that the pore diameter of the porous structure is 0.1 to 100 μm, more preferably 0.1 to 60 μm, still more preferably 0.1 to 40 μm, particularly preferably 1 to 20 μm. When the porosity and pore diameter of the porous structure are within the above-mentioned ranges, endothelialization at the end parts of the cover can be sufficiently promoted and a sufficient strength as a cover can be obtained.

It is preferred that the porous films made of the nonwoven fabric and woven fabric described above have a water permeability of 1×10¹ to 1×10⁵ ml/cm²/min, particularly preferably 1×10² to 1×10⁴ ml/cm²/min. When the water permeability of the porous film is within the above-mentioned range, endothelialization at the end parts of the stent cover can be sufficiently promoted.

Note that it is preferred the above-mentioned porous film is made from a material having biocompatibility. Also, it is more preferred that the film is made from a material having biodegradability or bioabsorbability. As such materials, those materials exemplified with respect to the film of the nonporous films can be advantageously used.

In the stent cover according to the present invention, the central part made of a nonporous film and the end parts made of a porous film may be made from the same material or from different materials. However, when they are made from different materials, it is required that they can be bonded to each other. The stent cover according to the present invention is preferably comprises a central part made of a nonporous film of silicone elastomer and each end parts made of a polyester nonwoven fabric. When the stent cover has the above-mentioned compositions, bonding between the central part and end parts is excellent, promotion of endothelialization at the end parts of the stent cover is sufficient, and its biocompatibility is excellent.

In the stent cover according to the present invention, bonding of the nonporous film at the central part to the porous films at the end parts can be preferably practiced by any known method. Examples of such method include fusion bonding, adhesion with an adhesive, fixing, suture with a thread or the like, it can be selected properly according to quality of a material.

Note that as for the bonded part between the central part and end parts of the stent cover, it is preferred that the parts are bonded such that they overlap at the boundary part each other. The parts are bonded as described above, no gap can occur therebetween and the parts are bonded securely. Here, as for the order of overlapping, as will be shown in Example described below, it is preferred that central part and end parts of the stent cover can be bonded in a state where they overlap one on another at their boundary parts such that central part overlaps on end parts (end parts is closely placed on the stent body covered by). Alternatively, when the central part and the end parts are bonded, edges of the parts may be bonded each other by adhesion with an adhesive, suture or the like, with out being overlapped them at the boundary part.

A drug may be added to the stent cover of the present invention for the purpose of preventing restenosis of body duct and promoting endothelialization at end portions of the stent. Such drug can't be limited as for its medicinal property, it can be selected properly depend on its need. It is preferred that such a drug is the one that principally has antithrombogenic effect, cell migration preventing effect, cell growth preventing effect, or endothelial cell growth promoting effect. More specifically, a carcinostatic agent such as paclitaxel, an immunosuppressive agent such as sirolimus, anti-platelet such as abciximab, phosphorylcholine, steroids, angiopeptin, forskolin, hirudin, iloprost, virus vectors and DNAs (bFGF, VEGF, HGF, TFPI, etc.) are exemplified. These may be added as single substance or as mixtures of a plurality of drugs.

In adding the above-mentioned drug to the stent cover, it is preferred that the drug is added as contained in a gel, as a mixture or in an encapsulated state from the point of view of handling properties. It is preferred that the gel used for this purpose is inactive to organism. A gel used for this purpose specifically includes gelatin, poly (2-methoxyethyl acrylate), chondroitin sulfate, hyaluronic acid, fibrin, etc. These may be used as single substance or as mixtures of two or more of them to form gel. It is more preferred that the gel has biodegradability or bioabsorbability.

Because of its end parts formed by a porous film, when a drug is added to the stent cover according to the present invention, the drug can be filled into pores of the porous film or air gaps of non woven fabric, whereby the stent cover according the present invention has an advantage in that it can carries an increased amount of drug as compared with the stent cover that is made of a nonporous film in its entirety.

In the stent cover according to present invention, portions that a drug is added thereto can be selected properly depend on medicinal property of the drug added to. A drug having a medicinal property whereof promotes endothelial cell growth, that is, a drug for promoting endothelialization, is preferably added to the stent cover covering inner side surface of the stent body in its entirety, that is a drug is added to both nonporous film of the central part and porous film of the end parts of such stent cover. By such addition, endothelialization can be promoted on the surface of the stent cover facing lumen of the stent in its entirety. On the other hand, a drug having a medicinal property whereof prevents hyperplasia or thrombosis is preferably added to end parts of the stent cover because hyperplasia and thrombosis susceptible to occur at the end portions of the stent.

The stent according to the present invention comprise a stent body and a stent cover(s). In the stent according to the present invention, a stent body has a cylindrical structure wherein open at both ends and longitudinally elongates between the both open ends having an outer side surface and an inner side surface. The cylindrical structure has a plurality of cutout parts on the side surface thereof communicating inner and outer side surfaces. The cylindrical structure is constructed so that with deformation of the cutout parts, the expansion and contraction in radial direction of the cylindrical structure is possible. In first embodiment of the stent according to the present invention, outer side surface and/or inner side surface of the stent body described above is covered by the stent cover(s) according to the present invention. The cutout parts can be cutout literally, or can be an opening formed by a linear member.

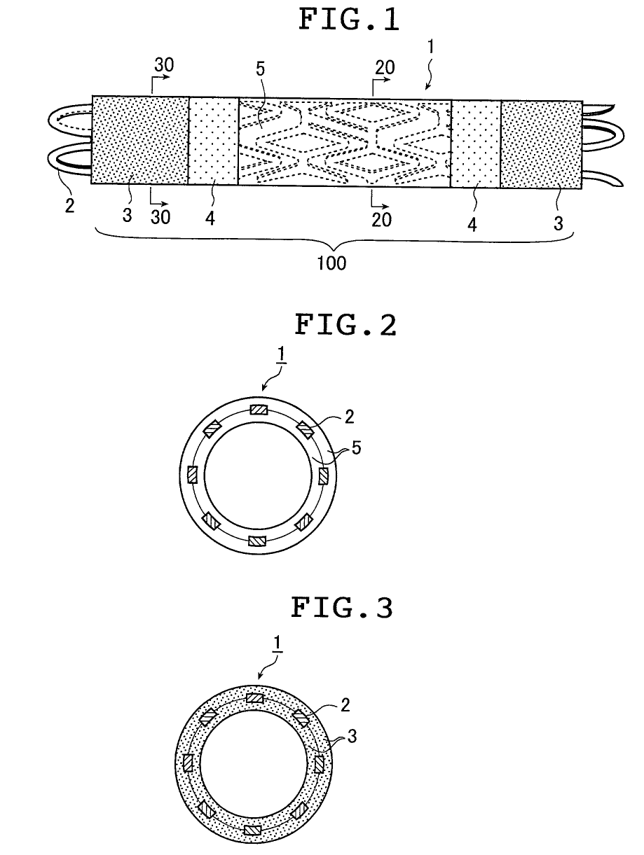

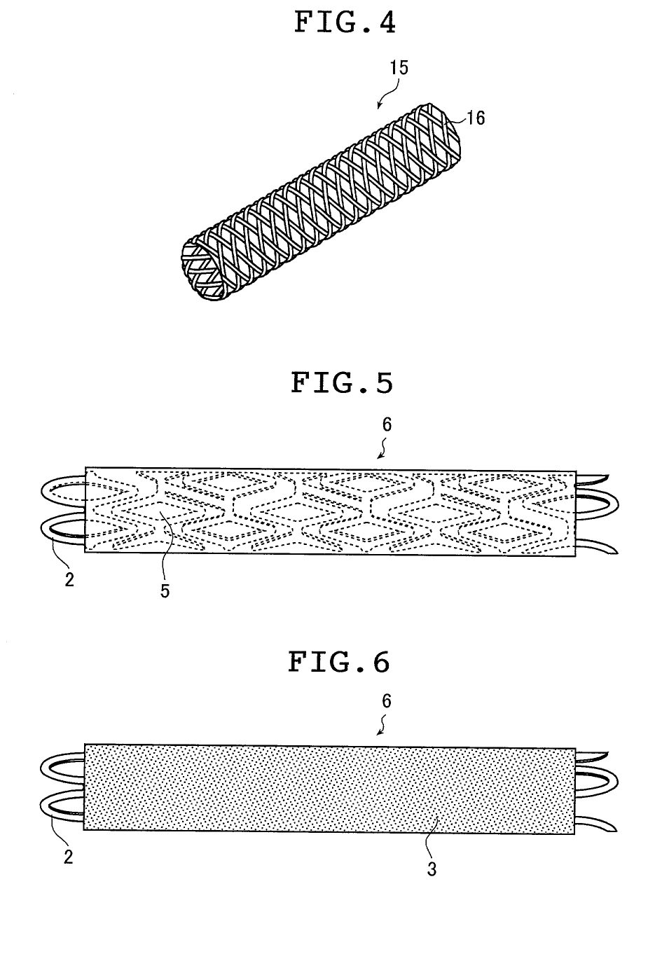

FIG. 1 is a side elevational perspective view showing a mode of construction of first embodiment of the stent according to the present invention. FIG. 2 is a cross sectional view taken along line 20-20 of the stent shown in FIG. 1. FIG. 3 is a cross sectional view taken along line 30-30 of the stent shown in FIG. 1. FIG. 4 is a perspective view showing a mode of stent body which is different in shape with that of FIG. 1. In FIG. 1, a stent body 2 wherein constitute a part of the stent 1 according to the present invention is in a form of a cylinder with its both ends opened like the stent body of FIG. 4. Inner and outer side surfaces of the stent body 2 are covered by the stent covers 100 according to the present invention. In FIG. 1, a central part 5 of the stent cover 100 is made of a nonporous film and its end parts 3 are made of porous films. Reference numeral 4 designates joint parts between the central part 5 made of a nonporous film and the end parts 3 made of a porous film. As shown in FIG. 2, at the central part of stent 1, inner and outer side surfaces of the stent body 2 are covered by nonporous films 5. As shown in FIG. 3, at the end parts of the stent 1, inner and outer side surfaces of the stent body 2 are covered by porous films 3.

The stent body 2 has a cylindrical structure wherein open at both ends and longitudinally elongates between the both open ends. The cylindrical structure has a plurality of cutout parts on the side surface thereof communicating inner and outer side surfaces. The cylindrical structure is constructed so that with deformation of the cutout parts, the expansion and contraction in radial direction of the cylindrical structure is possible. The stent body 2 is placed in the body duct such as vessels including blood vessels, bile ducts and the like to serve the structural support for the lumen. In FIG. 1, the stent body 2 has a mesh-like structure in overall shape having a plurality of cutout parts substantially rhomboid shaped on a side surface thereof communicating inner and outer side surfaces with each other, so that deformation of it by increasing and decreasing in radial direction of the stent body 2 is possible by application of stress thereto. Such cylindrical structures having a plurality of cutout parts on the side surface thereof communicating inner and outer side surfaces wherein is constructed so that with deformation of the cutout parts, the expansion and contraction in its radial direction is possible are exemplified hereinafter. FIG. 4 is a perspective view showing a cylindrical stent body 15 constructed by connecting plural elastic wire members 16 each other, the wires 16 are bent in spiral shape, cutout parts are constituted by clearance between each wire members. JP 09-215753 A and JP 07-529 disclose a cylindrical stent body constructed as shown in FIG. 4. In another example of the stent body, JP 08-502428 A and JP 07-500272 A disclose a cylindrical stent body constructed by connecting plural elastic wire members each other, the wire members are bent in zigzag, cutout parts are constituted by clearance between each wire members. JP 2000-501328 A discloses a cylindrical stent body which is formed of elastic wire members bent into a serpentine flat ribbon and wound in helix shape around a mandrel, cutout parts are constituted by clearance between each wire members. JP 11-221288 A discloses a stent body similar to the stent body as described in JP 2000-501328 A in its structure. JP 10-503676 A discloses a cylindrical stent body having a mesh-like structure whereof the cutout parts on a side surface is shaped like meander patterns. The stent body is different in shape with that of FIG. 1 in shape. JP 08-507243 A discloses a cylindrical stent body which is formed of elastic sheet member bent in spiral, cutout parts are constituted by clearance between each adjacent coil portions. JP 04-68939 B discloses plural cylindrical stent bodies having various structure including a cylindrical stent body wherein is constructed by forming elastic wire members in spiral and cutout parts are constituted by clearance between each adjacent spiral portions, a cylindrical stent body wherein is constructed by blading elastic wire members and cutout parts are constituted by clearance between each wire members and the like. A stent body 2 includes a leaf spring-like, a multiple helix-like, an irregular shaped tube-like, and the like besides the above-mentioned structure. And also, FIG. 2(a), (b) of JP 04-68939 A discloses a cylindrical stent body constructed by bending an elastic sheet member in convolute form. Such cylindrical stent body wherein has no cutout part on the side surface, but is constructed that the expansion and contraction in radial direction is possible can be used as the stent body according to the present invention. These all above-mentioned published work and pending patent applications cited herein are hereby expressly incorporated by reference in full.

As for the stent body, means to expand the placed stent body in its radial direction isn't limited. It may be a self-expanding type, wherein place in folded compact shape and by remove the force retaining such compact shape, expand in radial outward direction with its own restorative force. It also may be a balloon-expansion type, wherein is expanded in radial outward direction by an external force whereby placing a balloon inside the stent body and inflating the balloon.

Still more, a stent body isn't limited for the type placed in vessels including blood vessels. It may be the one placed at any stenosed portion inside body ducts (that is lumens), such as bile duct, to expand the lumens and serve the structural support for the lumens.

The material for the stent body includes polymer materials and metal materials. They are not particularly limited as long as they have rigidity and elasticity to a certain extent. However, it is preferably a material that has biocompatibility. More specifically, examples of the polymer material include polyolefins such as polyethylene and polypropylene; polyesters such as polyethylene terephthalate; fluorine-containing polymers such as polytetrafluoroethylene and tetrafluoroethylene-ethylene copolymer. Examples of the metal material include stainless steel, tantalum, titanium, nickel titanium alloy, tantalum titanium alloy, nickel aluminum alloy and the like. Among these, super-elastic alloys such as titanium nickel are preferred for a self-expanding type stent body. Above-mentioned materials for a self-expanding type stent body are superior in restorating force to its original shape. As for a balloon-expanded type stent body, it is preferred that it is less likely to restore its original shape once the stent is expanded, stainless steel and the like are preferred for. The production method of a stent body isn't limited and can be selected properly from any known method depend on its structure and material. For example, in the case of the above-mentioned mesh-like structure shown in FIG. 1, it can be produced by cutting out a pipe section made of the above-mentioned metal material having a required size and partially removing its side surface by machining, chemical etching, laser cutting or the like to form a plurality of cutout parts.

In first embodiment of the stent, material and producing method of the stent cover 100 are exemplified as described above.

However, FIG. 1 shows a mode wherein both inner and outer side surfaces are covered by the stent cover according to the present invention. Note that the stent according to the present invention isn't limited for the mode. In another mode of stent, either inner side surface or outer side surface of the stent body may be covered by the stent cover according to the present invention. In that mode, another side surface (the surface isn't covered by the stent cover according to the present invention) may be covered by the cover of prior art made from a porous film in its entirety or may be uncovered.

In the stent according to the present invention, it is preferred that both inner and outer side surfaces are covered by the stent covers according to the present invention. In the stent wherein both inner and outer side surfaces are covered by the stent cover according to the present invention, the stent cover covering outer side surface of the stent body reduces stimulation against a wall of body duct that the stent placed in, whereby hyperplasia of a lumen tissue can be prevented. The stent cover covering inner side surface of the stent body prevent that body fluid in the body duct contacts with the stent body directly, especially whereby prevents thrombosis when the body fluid is blood. And more, porous films constructing the end parts of the stent cover behave as a good anchorage for promoting endothelialization. On the other hand, a stent wherein either inner side surface or outer side surface of the stent body can be decreased the whole thickness of the stent (including wall thickness of the stent body and thickness of stent cover), whereby lumen of body duct wherein stent is placed can be enlarged. In the case that either inner side surface or outer side surface of the stent body is covered by the stent cover according to the present invention, it is preferred that outer side surface of the stent body is covered by the stent cover according to the present invention. When outer side surface of the stent body is covered by the stent cover according to present invention, growth and hyperplasia of a lumen tissue into inner lumen of the stent can be prevented and lumen where the stent is placed can be enlarged.

FIG. 1 shows a mode in the state the both end portion of stent body 2 exposed from the stent cover 100. A stent according to the present invention isn't limited for the mode. A stent body may be covered by the stent cover in its entirely (to its both end portions). The stent wherein the both end portions of the stent body are exposed from the stent cover is favored in promoting the endothealiazation at the ends of the stent and superior in fixation to the tissue. On the other hand, the stent wherein the stent body is covered by the stent cover in its entirely is superior in preventing an occurrence of hyperplasia at the lumen of the stent.

In the mode wherein both end portions of the stent body are exposed from the stent cover, the ratio of length of exposed portion to that of non exposed portion in the long axial direction is preferred 50:50 to 10:100, more preferably 30:70 to 10:100, still more preferably 20:80 to 10:100. Setting the ratio of exposed portion within above-mentioned range, it is favored in promoting the endothealiazation at the end portion of the stent and superior in fixation to the tissue.

A drug may be added to the stent cover 100 of the present invention for the purpose of preventing restenosis of body duct and promoting endothelialization at the end portoins of the stent. Drugs added to stent cover and means adding drugs to stent cover are exemplified as described above.

Method for covering inner side surface and/or outer side surface of stent body 2 with stent cover 100 isn't limited and can be selected from any known method as well as the stent cover 100 can maintain adequate coverage without detachment from the stent body 2 even if the stent body 2 may expand or contract in its radial direction and the method have no adverse effect on organism. For example, after coating the stent covers with a fluoric elastomer solution and evaporating the solvent, the stent covers 100 are contacted to the stent body 2, so that they may be bonded and fixed on the inner side surface and/or outer side surface of the stent body 2. And more, in the case that the stent cover is made of thermoplastic material, after placing the stent cover with closely contacted to inner side surface and/or outer side surface, the stent cover may be thermally fused to the stent body by heating.

In second embodiment of the stent according to the present invention, though outer side surface of the stent body is covered by the stent cover according to the present invention, at inner side surface of the stent body, only its both end portions are covered by porous films, its central portion is uncovered.

In second embodiment of the stent, because of outer side surface of the stent cover covered by the stent cover according to the present invention wherein central part of the stent cover is formed by nonporous film, the central portion of the stent has low water permeability, whereby infiltration of lumen cells from the body duct to inner luminal side of the stent body can be prevented and growth and hyperplasia of a lumen tissue into inner luminal side of the stent body can be prevented. On the other hand, because of each end portions of both outer side surface and inner side surface of the stent body covered by porous films, each end portions of the stent have high water permeability, whereby formation of smooth endothelialized surface from the vascular wall of a part in the vicinity of the cover to the surface of the stent cover facing the lumen of the stent can be promoted and it is superior in fixation to the tissue. And more, porous films covering the both end portions of inner side surface of the stent body behave as a good anchorage for promoting endothelialization.

In second embodiment of stent, the ratio of length of the central part to that of both end parts of the stent cover covering outer luminal wall are exemplified as described above with respect to first embodiment of the stent. And more, a length of porous films covering the both end portions of inner side surface is set in order the ratio of length of central portion which is uncovered to that of both end portions of stent body which are covered by porous films is similar to the ratio of length of the central part to that of the end parts of first embodiment of stent cover as described above.

And more, in second embodiment of the stent, porous films covering inner side surface of stent body may cover stent body with its both end portions covered in entirety or may cover stent body with its both end portions exposed. In the case that porous films cover the stent body with its both end portions exposed, the ratio of length of exposed portion to that of unexposed portion of ends of stent is exemplified as described above with respect to first embodiment of the stent.

In second embodiment of the stent, structure, materials and production methods of the stent body; materials and producing methods of nonporous film and porous film; methods for bonding between nonporous film and porous film are exemplified as described above with respect to the stent cover according to the present invention and first embodiment of the stent.

In second embodiment of the stent, a drug may add to a stent cover covering inner side surface of the stent body and/or porous films covering end portions of inner side surface of the stent body. Drugs added to stent cover and porous films, means adding drugs to stent cover and porous films are exemplified as described above.

### EXAMPLES

Hereinafter, the present invention will be described in more detail by way of examples.

### Example 1

In the instant example, a stent shown in FIG. 1 to FIG. 3, being first embodiment of the stent according to the present invention was formed by covering both an outer side surface and inner side surface of a cylindrical stent body 2 with a stent cover 100, bonding and fixing the stent cover 100 to the stent body 2. The cylindrical stent body 2 wherein opens at both ends having a diameter of 1.5 mm, a length of 20 mm and a thickness of about 120 μm, has a mesh-like structure in overall shape having a plurality of cutout parts substantially rhomboid shaped on a side surface thereof communicating inner and outer side surfaces with each other. The stent cover 100 of about 100 μm in thickness comprising the central part 5 made of a nonporous film and both end parts 3 made of a porous film (water permeability: 1100 (ml/cm²/min)). In FIG. 1, the central part 5 of the cover 100 is made of a nonporous film made of silicone elastomer having a length of about 10 mm and each of the end parts 3 of the cover 100 was made of a porous nonwoven fabric made of polyolefin having a length of about 5 mm. Two boundary parts 4 of the two covers overlapped one on another by about 2 mm such that the silicone elastmer film overlaps on the polyolefin nonwoven fablic (the polyolefin nonwoven fabric is closely placed on the stent body 2 covered by).

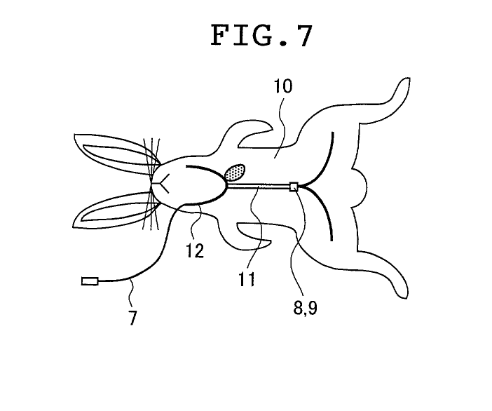

By using the stent thus formed, implantation tests into rabbit abdominal aorta were conducted. FIG. 7 is a diagram that illustrates the manner of implantation of the stent into rabbit abdominal aorta. As shown in FIG. 7, a stent 9 mounted on a balloon 8 of a balloon catheter 7 was introduced into a right common carotid artery 12 of a rabbit 10 and delivered to a abdominal aorta 11 of the rabbit 10 where hydraulic pressure of 10 atm was applied to the balloon 8 to expand it to a diameter of about 3 mm, thereby the stent 9 was implanted in the abdominal aorta 11 of the rabbit 10. After 4 weeks from the implantation, autopsy was conducted and pathological evaluations were done. As a result of pathological evaluations, no adherence of thrombi was noticed and smooth endothelialization over the surface of the stent cover 100 facing the lumen of the stent 1 was observed under way.

### Example 2

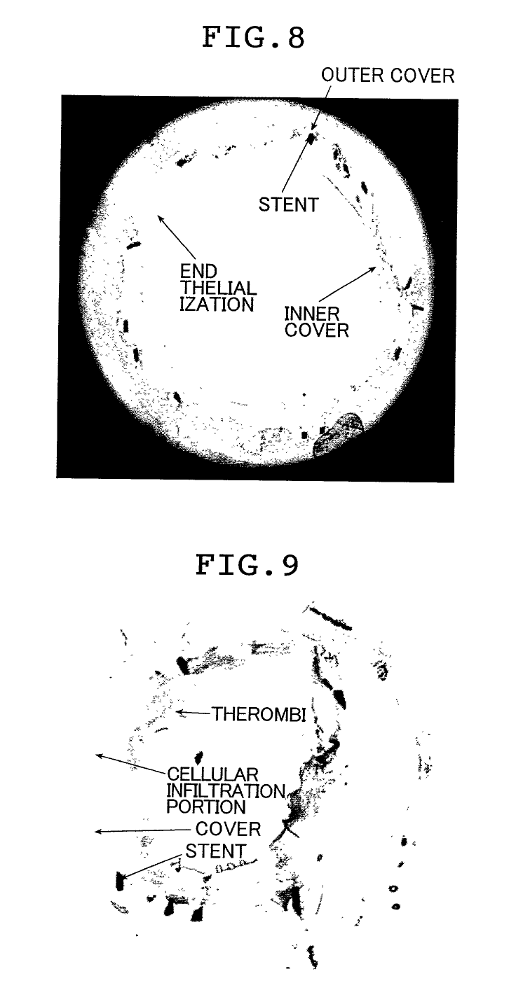

In the same manner as in Example 1, a stent being second embodiment of the stent according to the present invention was formed. However, unlike Example 1, at inner side surface of the stent body, only its end portions were covered by porous films and its central portion was uncovered. That is, in the stent of Example 2, its central part has only outer cover, its end portions have outer cover and inner cover. By using the stent thus formed, implantation tests into rabbit abdominal aorta and pathological evaluations were conducted. Each porous films covering end portions of inner side surface wall comprise a porous nonwoven fabric (water permeability: 1100 (ml/cm²/min)) made of polyolefin having a length of about 5 mm and a thickness of about 100 μm. FIG. 8 illustrates an image of pathological tissue seen from the upstream side of a cross-section vertical to the axis of the stent obtained by cutting a portion in the vicinity of an end part of the stent in the abdominal aorta 11 of the rabbit 10 having implanted therein the intravascular stent 9 of the Example. The picture was taken at a magnification of 20 folds. From FIG. 8, no adherence of thrombi was noticed and smooth endothelialization over the surface of the stent cover facing the lumen of the stent was observed under way.

### Comparative Example 1

In the same manner as in Example 2, a film of about 100 μm thick was covered as a cover on the outer side surface of a stent body having a diameter of 1.5 mm, a length of 20 mm and a thickness of about 120 μm and fixed to form an stent as shown in FIG. 5. However, unlike Example 1, a cover 5 of about 20 mm long was made of a nonporous film of silicone elastomer in its entirety. The stent thus formed was implanted in the abdominal aorta of a rabbit in the same manner as in Example above. After 4 weeks from the implantation, the animal was autopsied and pathological evaluation was done. FIG. 9 illustrates an image of a pathological tissue seen from the upstream side of a cross-section vertical to the axis of the stent obtained by cutting a portion in the vicinity of an end part of the stent in the abdominal aorta of the rabbit having implanted therein the stent of Comparative Example 1. The picture was taken at a magnification of 20 folds. From FIG. 9, no cell that passed through the cover portion and infiltrated into the lumen was observed. However, at the end parts of the cover, a state of cellular infiltration into the lumen was recognized, and also a state of forming thrombi there was observed.

### Comparative Example 2

A film of about 200 μm thick in total was covered as a cover on the inner and outer side surfaces of a stent having a diameter of 1.5 mm, a length of 20 mm and a thickness of about 80 μm and fixed to form an stent shown in FIG. 6. In FIG. 6, a cover 3 of about 20 mm in length was made of a porous film of polyolefin (water permeability: 350 (ml/cm²/min)) in its entirety. The stent thus formed was implanted in the abdominal aorta of a rabbit in the same manner as in the Example above. After 4 weeks from the implantation, the animal was autopsied and pathological evaluation was done. As a result, a state of cellular infiltration through the cover parts into the lumen was observed.

### INDUSTRIAL APPLICABILITY

When the stent cover of the present invention is used as a cover(s) for a stent body placed in body duct like the vessel, because of its central part made of a nonporous film, cellular infiltration from body duct into the inner luminal side of the stent body can be prevented. And more, because of its both end parts made of a porous film, formation of smooth endothelialized surface is promoted from the vascular wall of a part in the vicinity of the stent cover to the surface of the stent cover facing the lumen of the stent, whereby disturbance of blood flow at end portions of the stent is eliminated. In the stent of the present invention, because the inner side surface and/or outer side surface of the stent body covered by the stent cover of the present invention, or because the outer side surface of the stent is covered by the stent cover of the present invention and the end portions of the inner side surface of the stent body are covered by the porous films, formation of thrombi at end portions of the stent can be prevented, whereby a state of patency of lumen can be maintained for a long period of time.

1. A cylindrical stent cover covering a cylindrical stent body wherein open at both ends and longitudinally elongates between the both open ends having an inner side surface and an outer side surface, said stent cover covers the inner side surface and/or outer side surface of said stent body, said stent cover comprises a central part made of a nonporous film and the both end parts comprise a porous film.

2. A stent cover according to claim 1, wherein the end parts of said cover comprises a nonwoven fabric.

3. A stent cover according to claim 1, wherein said nonporous film of the central part and said porous film of the end parts comprise a material having biocompatibility.

4. A stent cover according to claim 1, wherein said nonporous film of the central part and said porous film of the end parts comprise a material having biodegradability or bioabsorbability.

5. A stent cover according to claim 1, wherein further a drug for preventing restenosis of a stent-implanted vessel or promoting endothelialization at the end parts of the stent is added to said stent cover.

6. A stent cover according to claim 5, wherein said drug has at least one of effects selected from the group consisting of antithrombotic effect, prevention of cell migration, prevention of cell growth, and promotion of endothelial cell growth.

7. A stent cover according to claim 5, wherein said drug is added in a state where it is contained in gel.

8. A stent cover according to claim 7, wherein said gel is inactive to a living organism.

9. A stent cover according to claim 7, wherein said gel has biodegradability or bioabsorbability.

10. A stent comprise a stent body wherein open at both ends and longitudinally elongates between the both open ends having an inner side surface and an outer side surface, a stent cover(s) according to claim 1.

11. A stent comprises a stent body wherein open at both ends and longitudinally elongates between the both open ends having an inner side surface and an outer side surface, a stent cover according to claim 1 wherein covers said outer side surface, a porous film wherein covers both end portions of said inner side surface of the stent body but doesn't cover the central portion of said inner side surface of the stent body.
